(19) United States
(12) Patent Application Publication
(10) Pub. No.: US 2022/0226024 A1
(43) Pub. Date: Jul. 21, 2022

(54) BONY FUSION SYSTEM WITH POROUS MATERIAL REGIONS

(71) Applicant: Osseus Fusion Systems, Dallas, TX (US)

(72) Inventor: Chase D. Tipping, Dallas, TX (US)

(21) Appl. No.: 17/151,153

(22) Filed: Jan. 16, 2021

(51) Int. Cl. A61B 17/70; A61F 2/44; A61L 27/36

(52) U.S. Cl. CPC A61B 17/7032; A61F 2/4455; A61F 2002/30622; A61L 27/3608; A61B 17/7002

(57) ABSTRACT

Embodiments of bony region stabilization and fusion constructs are described generally herein including bony fixation elements including porous, bone fusion enabling or promoting regions or osteo conductive enabling or promoting regions. Other embodiments may be described and claimed.

### TECHNICAL FIELD

Various embodiments described herein relate generally to stabilizing, promoting fusion between, and fusing mammalian bony segments, including systems and methods for stabilizing adjacent level mammalian bony segments.

### BACKGROUND INFORMATION

It may be desirable to stabilize and fuse one or more adjacent level bony segments; the present invention provides such stabilization and fusion.

### BRIEF DESCRIPTION OF THE DRAWINGS

FIG. 1A is a simplified posterior diagram of mammalian bony segment where the invention may be employed according to various embodiments.

FIG. 1B is a simplified posterior diagram of mammalian bony segment with decorticated regions where the invention may be employed according to various embodiments.

FIG. 2A is an isometric image of a mammalian bony segment adjacent level stabilization and fusion promoting system according to various embodiments.

FIG. 2B is a front side image of a screw of a mammalian bony segment adjacent level stabilization system according to various embodiments.

FIG. 2C is an enlarged image of section AA shown in FIG. 2A according to various embodiments.

FIG. 2D is an enlarged image of section CC of section AA shown in FIG. 2C according to various embodiments.

FIG. 2E is an enlarged image of another section CC of section AA shown in FIG. 2C according to various embodiments.

FIG. 2F is an enlarged image of section BB shown in FIG. 2A according to various embodiments.

FIG. 3A is a simplified diagram of mammalian bony segment adjacent level stabilization and fusion architecture shown in FIG. 2A coupled to a mammalian bony segment shown in FIG. 1A according to various embodiments.

FIG. 3B is a simplified diagram of mammalian bony segment adjacent level stabilization and fusion architecture shown in FIG. 2A coupled to a mammalian bony segment with decorticated regions shown in FIG. 1B according to various embodiments.

FIG. 4A is an isometric image of another mammalian bony segment adjacent level stabilization and fusion promoting system according to various embodiments.

FIG. 4B is an isometric image of a rod with an empty bone graph compartment of a mammalian bony segment adjacent level stabilization system shown in FIG. 3A according to various embodiments.

FIG. 4C is an isometric image of a rod with bone graph material embedded in its compartment of a mammalian bony segment adjacent level stabilization system shown in FIG. 4B according to various embodiments.

FIG. 5A is a simplified diagram of mammalian bony segment adjacent level stabilization and fusion architecture shown in FIG. 4A coupled to a mammalian bony segment shown in FIG. 1A according to various embodiments.

FIG. 5B is a simplified diagram of mammalian bony segment adjacent level stabilization and fusion architecture shown in FIG. 4A coupled to a mammalian bony segment with decorticated regions shown in FIG. 1B according to various embodiments.

### DETAILED DESCRIPTION

Human structures such as adjacent bony elements (such as human vertebrae) separated by non-bony elements (such as disc nucleus) may become unstable due to injury or natural processes including aging. To help stabilize such human structures, one or more adjacent bony elements may be coupled together via a fusion promoting system. Apparatus may also be placed between one or more adjacent bony elements to help stabilize their relationship. In an embodiment, one or more adjacent bony elements may also desirably become fused over time to ensure stabilization of the related human structure including such elements. In an embodiment, a fusion promoting system used or employed to stabilize one or more adjacent bony elements may also become part of the fusion construct of the human structure.

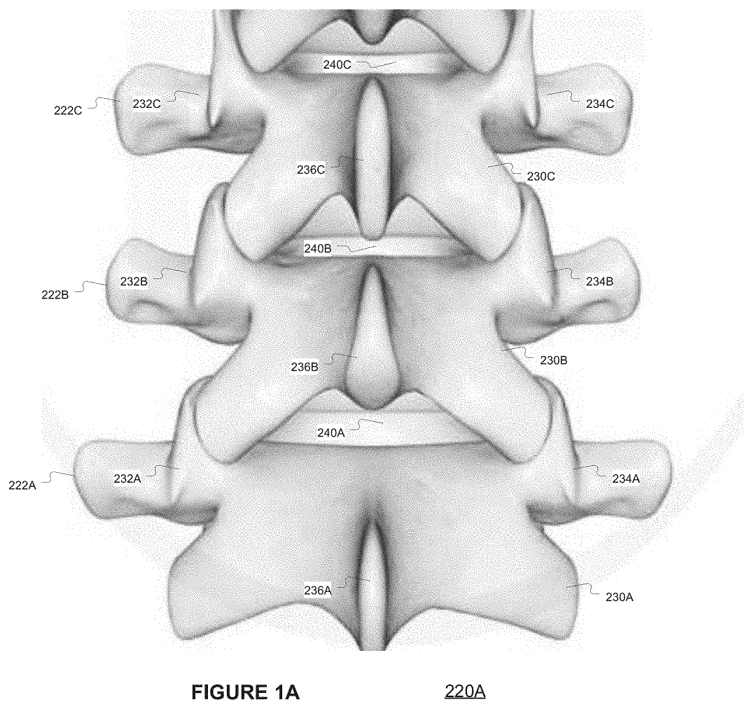

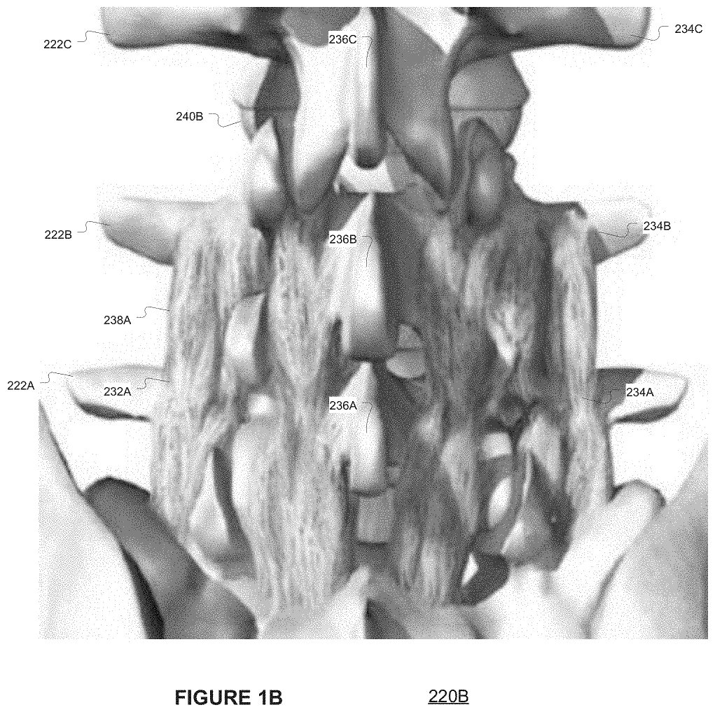

FIG. 1A is a simplified posterior diagram of a human structure including a mammalian bony segment 220A where embodiments of the invention may be employed according to various embodiments. The bony segment 220A includes adjacent bony structures 222A-C. In an embodiment one or more bony regions or structures 222A, 222B, 222C may be separated by one or more non-bony elements 240A, 240B, 240C. In an embodiment, the bony regions 222A, 222B, 222C may be vertebra separated by spinal discs 240A, 240B, 240C in a cervical, thoracic, or lumbar region of a mammal including a human. In embodiment, each bony element 222A-C may include a pedicle pair 232A-C, 234A-C, and a dorsal spinous process 236A-C.

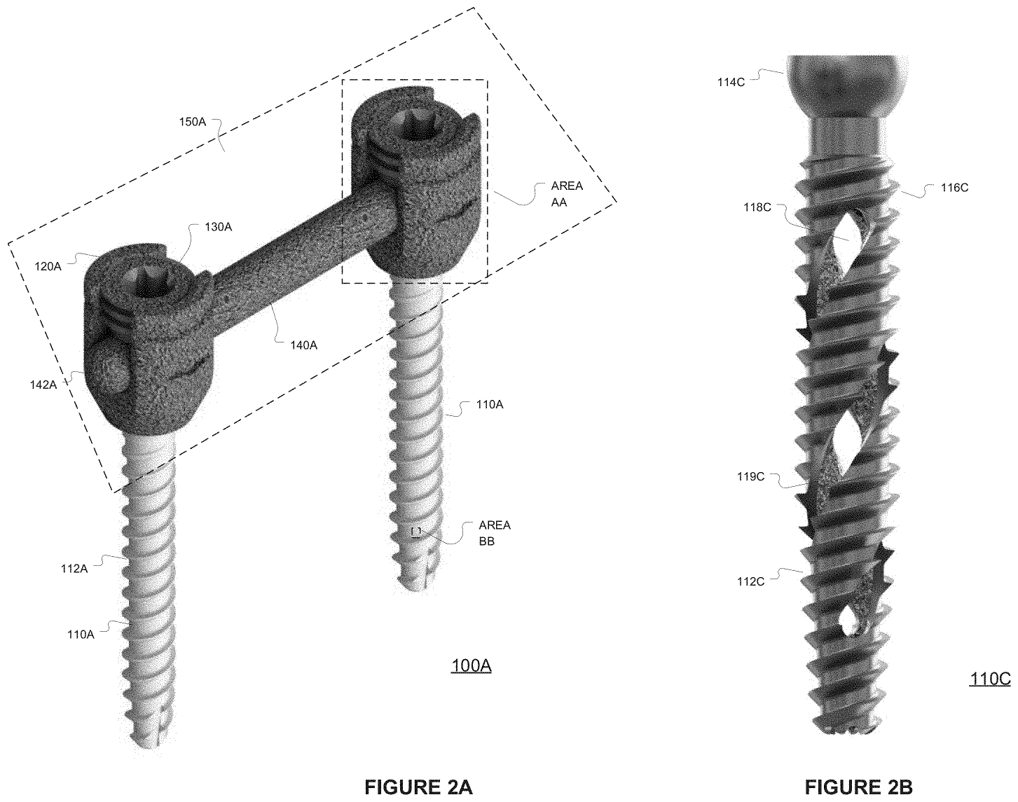

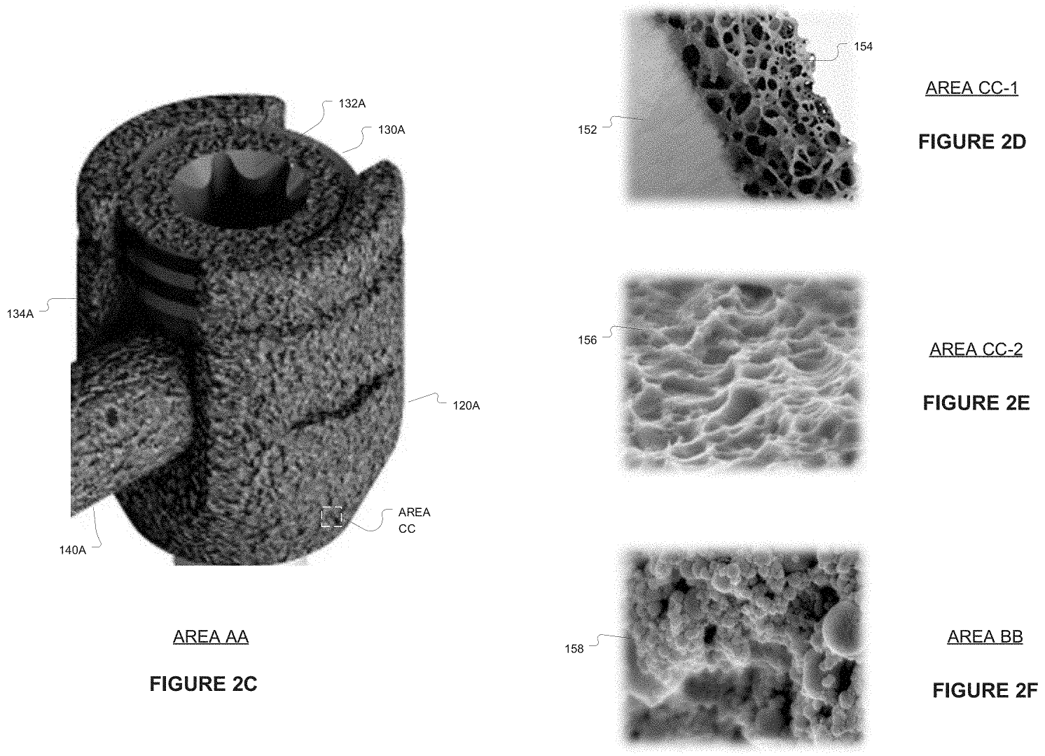

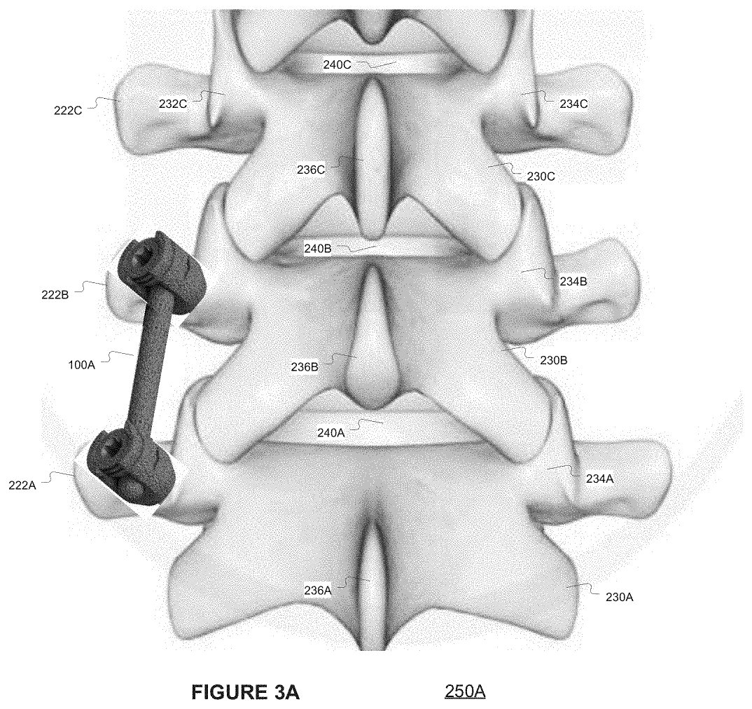

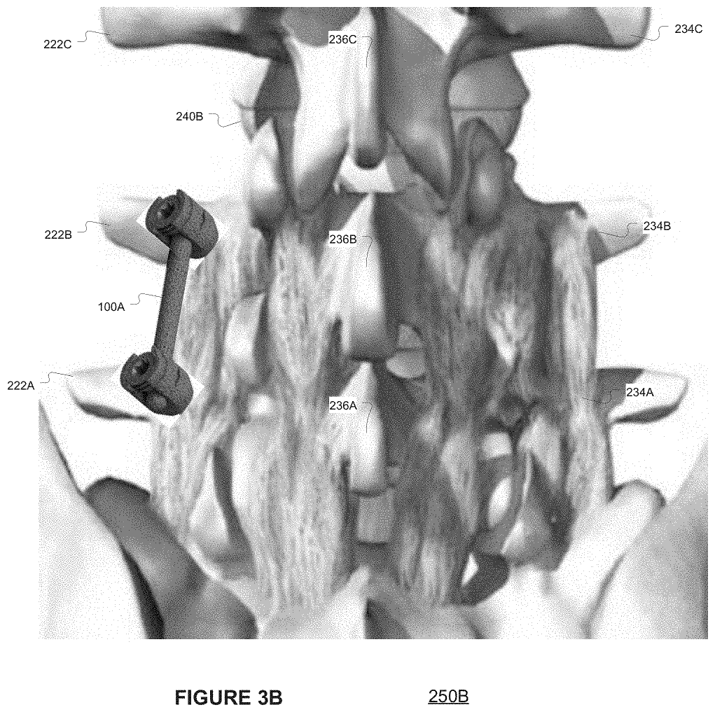

FIG. 1B is a simplified posterior diagram of a mammalian bony segment 220B with decorticated regions 238A where embodiments of the invention may be employed according to various embodiments. Regions 238A may be decorticated to aid in the possible fusion of adjacent bony elements 222A-C. In an embodiment, a mammalian bony segment adjacent level stabilization and fusion promoting system 100A-B (shown in FIGS. 2A-3B) may be employed in segments 220A-B.

FIG. 2A is an isometric image of a mammalian bony segment adjacent level stabilization and fusion promoting system 100A that may be employed in the bony segments 220A, 220B as shown in FIGS. 3A-B according to various embodiments to couple bony elements 222A-B via their pedicles 232A-B. As shown in FIG. 2A, a fusion promoting system 100A may include several apparatus including bone engaging members 110A that are coupled together via an engaging member coupling system 150A, 150B. The coupling system 150A may securely and fixably couple a plurality of bone engaging members 110A where the bone engaging members 110A may be securely and fixably coupled to separate bony segments. The coupling system 150A, 150B may include a cross member 140A, 140B that is coupled to the bone engaging members 110A via a lockable coupler 120A, 120B and a locking mechanism 130A. The locking mechanism 130A may hold the cross member 140A, 140B, bone engaging member 150A, 150B and lockable coupler 120A, 120B in a fixed relationship when engaged.

In an embodiment, the bone engaging members 110A, 110B, 110C may be screws such as pedicle screws 110A. The coupling system 150A may fixably couple two or more screws via a cross member 140A, 140B that is coupled to the bone engaging members 110A via a lockable coupler 120A, 120B and a locking mechanism 130A. The locking mechanism 130A may hold the cross member 140A, 140B, bone engaging member 150A, 150B and lockable coupler 120A, 120B in a fixed relationship when engaged.

In an embodiment, the coupling system 150A, 150B lockable coupler 120A, 120B include rod receiving heads 120A, 120B. The locking mechanism 130A, 130B may include set screws 130A, 130B. The cross member 140A, 140B may include rods 140A, 140B. In an embodiment, each rod receiving head 120A, 120B may engages screw 110A, 110B, 110C such as via a polyaxial head 114C.

In an embodiment, two pedicle screws 110A, 110B, 110C each engaging a bony element may be coupled together via a coupling system 150A, 150B. The coupling system 150A, 105B may include rod receiving heads (lockable coupler) 120A, 120B, set screws (locking mechanism) 130A, 130B, and a rod (cross member) 140A, 140B. The rod receiving heads 120A, 120B may include set screws 130A, 130B than enable a rod 140A, 140B end 142A to be secured to the head 120A, 120B and thus a pedicle screw 110A, 110B, 110C.

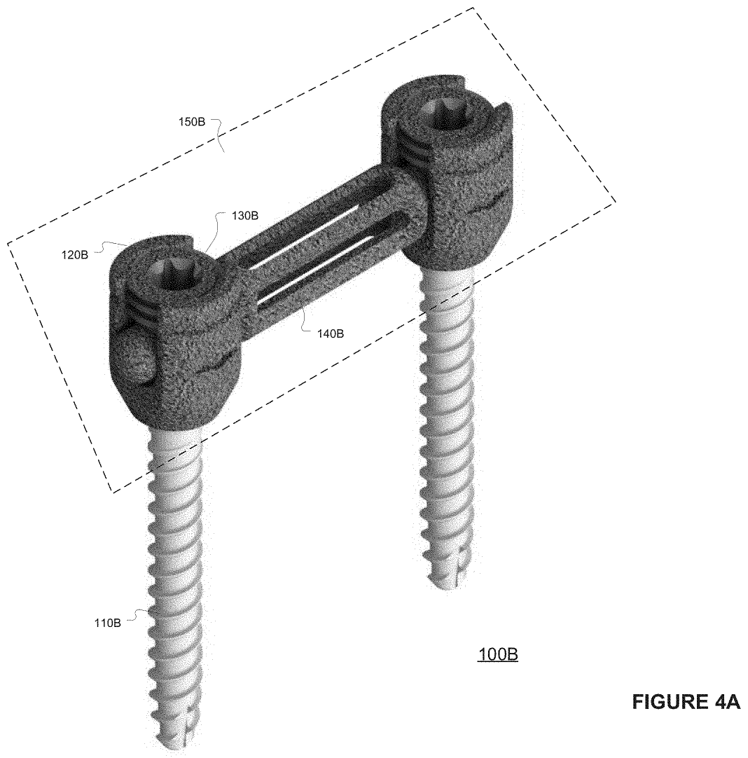

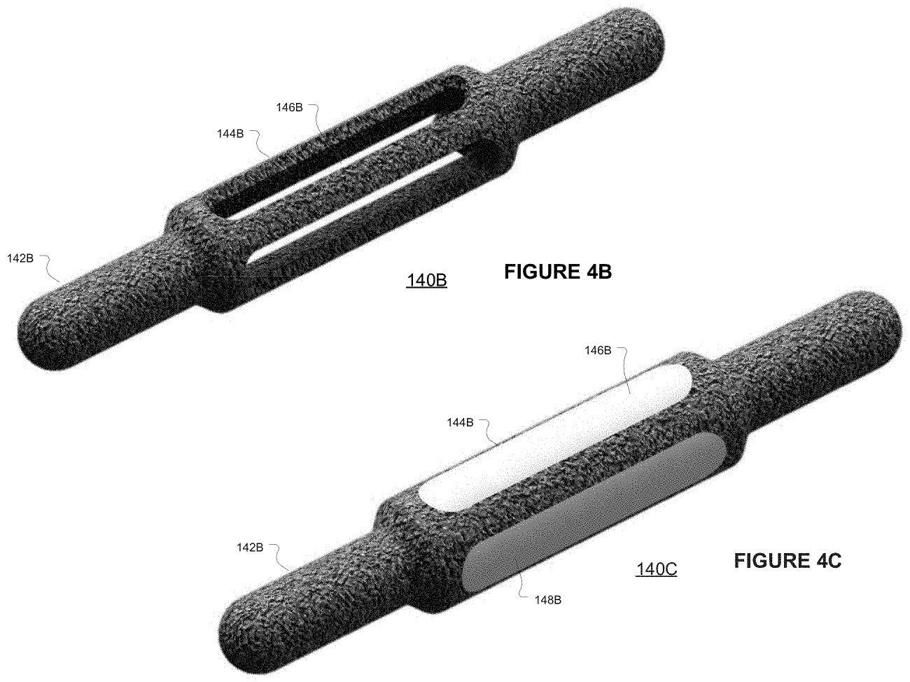

In an embodiment, portions of the fusion promoting system 100A apparatus may include porous regions such as shown in FIGS. 2D-2F. In an embodiment, any of or all of the pedicle screws 110A, 110B, 110C may include porous regions or osteo conductive regions as shown in FIGS. 2A, 2B, 4A. In an embodiment, any of or all of the coupling system 150A, 150B may include porous regions or osteo conductive regions as shown in FIG. 2A, 4A. In an embodiment, any of or all of the bone engaging members 110A 110B, 110C, the lockable couplers 120A, 120B, the locking mechanisms 130A, 130B, and the cross members 140A, 140B may include porous regions or osteo conductive regions as shown in FIG. 2A, 4A.

In an embodiment, a majority or all of the bone engaging member (pedicle screw's) 110A shaft 112A surface area may include porous regions or osteo conductive regions. In an embodiment, a majority or all of the cross members (rod's) 140A surface area may include porous regions or osteo conductive regions. In an embodiment, a majority or all of the lockable coupler (rod receiving heads') 120A surface area may include porous regions or osteo conductive regions other than the thread receiving area (that mate with locking mechanism (set screws) 130 threads). In an embodiment, a majority or all of the locking mechanism (set screws') 130A surface area may include porous regions or osteo conductive regions other than its thread (that mate with lockable coupler (rod receiving heads') 102 thread receiving areas).

As shown in FIG. 2B, a bone engaging member herein after pedicle screw 110A, 110C may include a shaft 112A, 112C and a polyaxial head 114C. The shaft 112A, 112C may include a thread 116C. In an embodiment, the pedicle screw 110C shaft 112C may include one or more fenestrations or slots 118C creating an opening in the shaft 112C. The fenestrations or slots 118C may enable or promote bony fusion between a pedicle screw 110C and bony elements 222A-C where the pedicle screw 110C may be employed as shown in FIGS. 3A and 3B. In an embodiment, the inside of the fenestrations 118C may include porous sections 119C that may aid or promote bony fusion or conduction.

In an embodiment, the portions porous regions of a pedicle screw 110A and coupling system 150A, 150B (including a lockable coupler hereinafter receiving head 120A, a locking mechanism hereinafter set screw 130A, and a cross member hereinafter rod 140A) may be formed during production of the each. FIG. 2C is an enlarged image of section AA shown in FIG. 2A according to various embodiments. As shown in FIGS. 2A and 2C in an embodiment not all portions of the fusion promoting system 100A may include porous regions. As shown in these figures, the set screw 130A tool interface 132A and threads or flange 134A may not include porous regions. In another embodiment, all or a portions of the screw 110A and the set screw 130A tool interface 132A and threads or flange 134A may include porous regions.

FIG. 2D is an enlarged image of an embodiment CC-1 of a porous region area CC of section AA of the rod receiving head 120A shown in FIG. 2A according to various embodiments. In an embodiment, the porous region CC-1 may include a solid metal or alloy 152 covered with a porous 3-D metal surface 154. In an embodiment, the metal 152 and porous metal surface 154 may be titanium and all or a portion may be formed via 3-D printing.

FIG. 2E is an enlarged image of another embodiment CC-2 of section CC of section AA shown in FIG. 4A according to various embodiments. Porous region CC-2 may be a surface 156 of a material that is formed by etching the material. In an embodiment, sections or the entire surface of screws 110A may be covered by a bone conduction material such as hydroxyapatite. FIG. 2F is an enlarged image of section BB of pedicle screw 110A shaft 112A shown in FIG. 2A according to various embodiments showing the coating of the surface 158 with a bone conduction material.

The porous regions of fusion promoting system 100A may have sizing and shaping similar to trabecular bone regions to aid the bone conduction and fusion of the regions to bony elements 222A-C so the fusion promoting system 100A provides stabilization during a fusion process and becomes part of the bony fusion in an embodiment. cancellous bone, is typically found at the ends of long bones and within the vertebral bodies. Trabecular bone regions may higher porosity with more space resembling a lattice-like structure than other bone regions such as cortical bone where individual trabeculae of humans may be about 500-200 μm thick. The spacing between walls may be about 150 to 300 μm and with wall thickness of about 50-100 μm for the porous regions in an embodiment. The fusion promoting system 100A may aid in the fusion process by creating more regions that enable bony fusion.

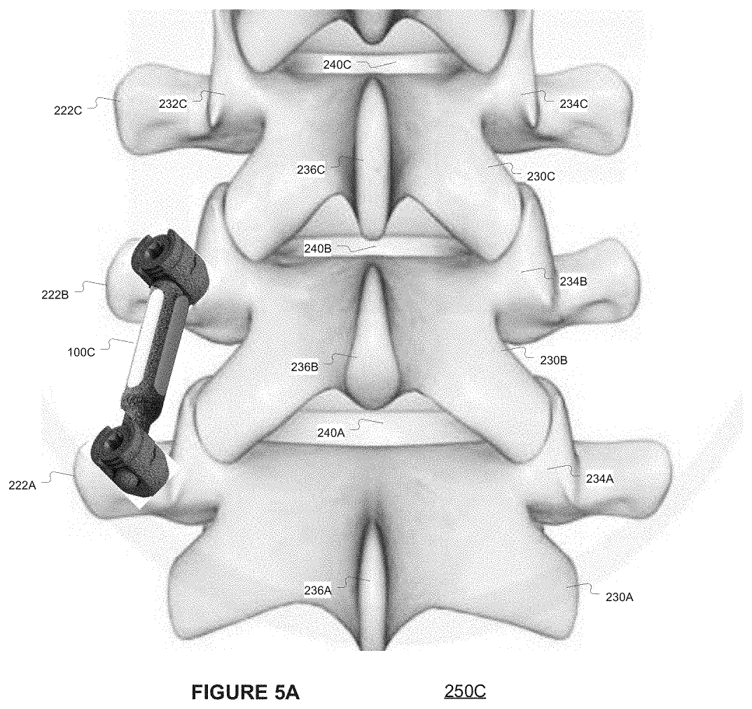

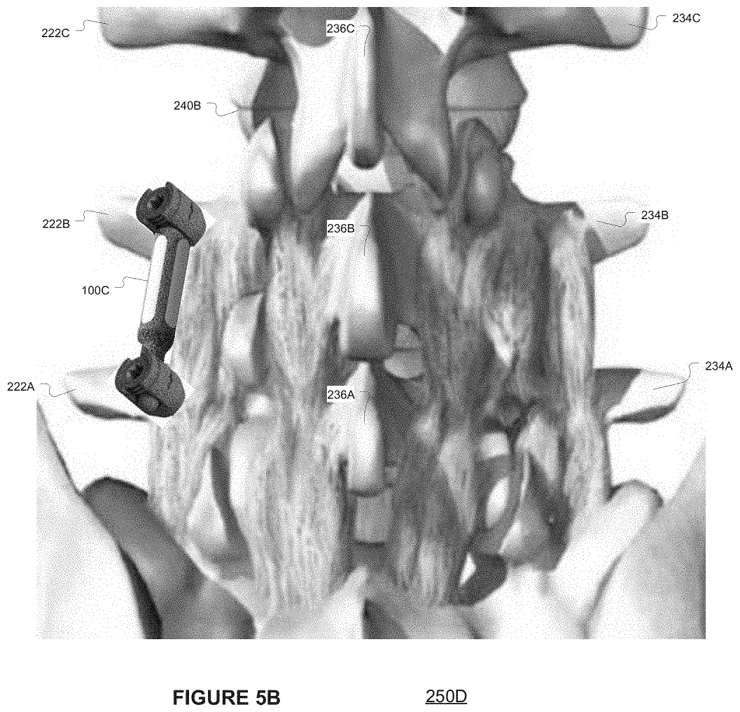

FIG. 4A is an isometric image of another mammalian bony segment adjacent level stabilization and fusion promoting system 100B that may be employed in the bony segments 220A, 220B as shown in FIGS. 5A-B according to various embodiments to couple two bony elements 222A-B via their pedicles 232A-B. As shown in FIG. 4A, a fusion promoting system 100B may include several pedicle screws 110B coupled together via a coupling system 150B including rod receiving heads 120B, set screws 130B, and a cavity rod 140B. Fusion promoting system 100B is similar to system 100A except coupling system 150B includes a rod 140B with open space 142B where material such as autogenous bone, bone matrix, or other osteo conductive material may be placed as shown in FIG. 4C. The system 100B cavity rod 140B is shown in more detail in FIGS. 4B and 4C may include arms 144B between ends 142B that form a cavity 146B (empty in FIG. 4B and filled with autogenous bone, bone matrix, or other osteo conductive material 148B in FIG. 4C). In an embodiment, all or a portion of the rod 140B may be covered with porous, bone fusion or osteo conductive enabling or promoting regions. The cavity 146B may be filled with autogenous bone, bone matrix, or other osteo conductive material 148B in an embodiment.

In an embodiment the systems 100A, 100B may be comprised of metal, metal polymer, ceramics, polymers, and combinations thereof. The polymers may include PEEK (Polyether ether ketone) in an embodiment. The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus, the following claims are hereby incorporated into the Detailed Description, with each claim standing on its own as a separate embodiment.

What is claimed is:

1. A fusion promoting system for stabilizing and promoting fusion of adjacent bony segments, the system including:
a pair of bone engaging members, each member configured and shaped to be securely and fixably couplable to a separate bony segment of the adjacent bony segments; and
a coupling system configured to securely and fixably couple the pair of bone engaging members, a majority of the surfaces of the coupling system including porous regions that aid or promote bony fusion or conduction.

2. The fusion promoting system of claim 1, the coupling system including:
a pair of lockable couplers each configured to engage one of the pair of bone engaging members and including one of internal and external threads; and
a pair of locking mechanisms each including threads to engage one of the pair of lockable couplers one of internal and external threads,
wherein the pair of lockable couplers surfaces include porous regions other than the one of internal and external threads and the pair of locking mechanism surfaces include porous regions other than the threads,
wherein the porous regions aid or promote bony fusion or conduction.

3. The fusion promoting system of claim 2, wherein the pair of bone engaging members surfaces include osteo conductive regions that aid or promote bony fusion or conduction.

4. The fusion promoting system of claim 2, the coupling system further including a cross member configured to be coupled to the pair of bone engaging members via the pair of lockable couplers, the cross member entire surface including porous regions that aid or promote bony fusion or conduction.

5. The fusion promoting system of claim 4, wherein each locking mechanism is configured to hold the cross member, one of the pair of bone engaging members, and one of the pair of lockable couplers in a fixed relationship when operatively engaged.

6. The fusion promoting system of claim 5, wherein each of the pair of bone engaging members is a bone screw.

7. The fusion promoting system of claim 6, wherein the lockable couplers are rod receiving heads, the locking mechanisms are set screws, and the cross members are rods and each rod receiving head is further configured to engage one of pair of bone screws head.

8. The fusion promoting system of claim 6, wherein each of the pair of bone screws includes a shaft with a thread where the shaft includes a fenestration creating an opening in the shaft.

9. The fusion promoting system of claim 6, wherein the inside of the fenestrations includes include porous sections that aid or promote bony fusion or conduction.

10. The fusion promoting system of claim 4, wherein the porous regions of each element are formed during production.

11. The fusion promoting system of claim 10, wherein each element is formed of a solid metal and porous 3-D metal surfaces forming the porous regions.

12. The fusion promoting system of claim 11, wherein the metal is titanium and the elements including porous regions are formed via 3-D printing.

13. The fusion promoting system of claim 10, wherein each element is formed of an alloy and porous 3-D alloy surfaces forming the porous regions.

14. The fusion promoting system of claim 4, wherein the porous regions of each element are formed by etching the element's surface.

15. The fusion promoting system of claim 6, wherein each of the pair of screws includes a shaft whose surface is covered by a bone conduction material.

16. The fusion promoting system of claim 4, wherein the porous regions of each element are formed to have sizing and shaping similar to trabecular bone regions of the adjacent bone segments.

17. The fusion promoting system of claim 16, wherein the porous regions of each element spacing between walls is from 150 to 300 μm and wall thickness is from 50-100 μm.

18. The fusion promoting system of claim 7, wherein the rod includes a cavity sized to be fillable with one of autogenous bone, bone matrix, and other osteo conductive material.

19. The fusion promoting system of claim 18, wherein the rod includes a plurality of arms between ends that form the cavity.

20. The fusion promoting system of claim 19, wherein the cavity is sized to be fillable with autogenous bone.
